(19) United States
(12) Patent Application Publication
(10) Pub. No.: US 2013/0072940 A1
(43) Pub. Date: Mar. 21, 2013

(54) POSITION OF HIP JOINT PROSTHESES

(71) Applicants: Andrew Joseph Stanley Dawood, London (GB); Stephen Robin Richards, London (GB); Alister James Hart, London (GB)

(72) Inventors: Andrew Joseph Stanley Dawood, London (GB); Stephen Robin Richards, London (GB); Alister James Hart, London (GB)

(21) Appl. No.: 13/636,818

(22) PCT Filed: Mar. 24, 2011
(86) PCT No.: PCT/GB2011/050601

§ 371 Date: Nov. 30, 2012

(30) Foreign Application Priority Data
Mar. 24, 2010 (GB) 1004878.3

(51) Int. Cl. A61B 17/56

(52) U.S. Cl. 606/91; 606/86 R

(57) ABSTRACT

Apparatus for guiding a shaping tool for shaping an acetabulum comprises locating means (40) arranged to be located in the acetabulum, guide means (50) for guiding the shaping tool, and support means (80) for supporting the guide means, wherein the guide means is arranged to locate against the locating means in a target position, and the support means is arranged to support the guide means in the target position after removal of the locating means.

### FIELD OF THE INVENTION

The present invention relates to hip joint prostheses used for hip joint replacement and hip joint resurfacing, and in particular to

### BACKGROUND TO THE INVENTION

Hip replacement or resurfacing operations are a common treatment for arthritic hip joints. Revision replacements are also relatively common in view of the large numbers of primary operations.

During the replacement or resurfacing procedure a joint prosthesis is inserted, having femoral and acetabular components. The longevity of the reconstruction and joint prosthesis, and the health of the surrounding tissues may be adversely affected by inappropriate positioning of the components of the prosthesis, leading to inappropriate mechanical loading, adverse wear and tear, loss or stability and biological complications.

Positioning problems are more often associated with the acetabular component; equally, correctly positioning the acetabular component appears to favour a longer lasting outcome, as it helps to provide ideal contact between the femoral and acetabular components, and ideal load distribution within, and optimised wear of, the two parts.

Access to the acetabulum is a somewhat invasive surgical intervention, particularly if the head of the femur is preserved, as is the case with ‘resurfacing’ procedures. Without wide exposure of the area, it can be difficult to adequately orientate the prosthetic components, particularly the acetabular component. However, post-operative healing and rehabilitation is improved by minimising surgical exposure of the acetabulum.

It is therefore desirable that any device intended to increase accuracy should not necessitate unnecessary exposure of the pelvis.

Various approaches including robotic or image guided navigation have been used in an attempt to improve spatial awareness and help to accurately position the prosthetic components.

Such techniques require preoperative planning using 3D imaging of the pelvis to plan an ‘ideal’ position for the acetabular component, but rely upon the availability of sophisticated apparatus.

The use of rapid prototyping to fabricate bone supported cutting or drilling guides, as used in other regions, e.g. the knee or in dental implant surgery, is not readily applicable to the acetabulum. In order to seat a rapid prototyped guide over the acetabulum for preparation of the acetabulum, a wide exposure of the region would be required in order to robustly seat an accurately positioned guide. This is because seating a drilling guide into the acetabulum itself would not be successful, as it is the acetabulum itself, which needs to be prepared for the prosthesis.

### SUMMARY OF THE INVENTION

The present invention provides a guided approach to surgical placement of the acetabular component (AC) of a joint prosthesis, in which 3D imaging and software may be used to define an ‘ideal’ angulation for the component and image the geometry of the acetabulum. The desired trajectory and depth of the AC may be transferred to the operative field by the use of a custom-made or customised ‘acetabular assembly’ which may fit within the acetabulum, projecting a guiding rod, which is used to transfer the relationship to a suitable reaming guide which is fixated into the Ilium, or other mechanically stable frame of reference by means of an ‘anchorage assembly’. After fixation, the acetabular assembly can be removed to allow access for reaming and implantation. The fixated guide is then, in turn, used to achieve accurate surgical preparation of the acetabulum and implant positioning to the planned position.

The present invention therefore provides apparatus for guiding a shaping tool for shaping a bone, or a part of a bone, for example an acetabulum, the apparatus comprising locating means arranged to be located on the bone, for example in the acetabulum, guide means for guiding the shaping tool, and support means for supporting the guide means. The guide means may be arranged to locate against the locating means in a target position. The support means may be arranged to support the guide means in the target position, either before or after removal of the locating means, or both.

The locating means may comprise a body portion arranged to fit against a surface on the bone, for example within the acetabulum. The locating means may comprise a locating portion arranged to locate, for example, in the acetabular notch.

The locating means may include a support guide, which may be separate from or integral with the locating portion, arranged to guide location of the support means. For example it may be arranged to guide location of the guide means. For example the locating means may comprise a removable locating member against which the guide means is arranged to be located.

The locating member and the body portion may comprise removable connection means arranged to locate the locating member relative to the body portion. The body portion may have a recess therein arranged to receive the locating member. The recess may extend part way through the body portion, or completely through the body portion forming an aperture therethrough.

The locating means may comprise a body and a plurality of spacer elements arranged to be supported on the body and to locate against the bone, for example in the acetabulum.

Indeed the present invention further provides apparatus for locating an object in a desired position relative to a bone, the apparatus comprising a support body, locating means arranged to locate the object relative to the support body, and a plurality of spacer members arranged to be attached to the support body. The spacer members may each have a contact surface thereon arranged to contact the bone thereby to locate the support body relative to the bone.

The spacer elements may be each formed from a flat sheet of material, for example by laser cutting. Alternatively they may comprise rods or blocks or other shaped members. The apparatus may further comprise locking means arranged to lock the spacer elements in fixed positions relative to the body.

The support means may include an anchor arranged to be anchored in the bone, for example to the pelvis, at a position spaced apart from the region to be shaped.

The support means may comprises a plurality of components which are movable relative to each other and locking means arranged to lock the components in a fixed position relative to each other. Alternatively it may comprise a series of interlocking members at least one of which can be selected from a set of different members so as to provide location of the guide means in a target position.

The present invention further provides a system for producing an apparatus according to the invention as described above, the system comprising processing means arranged to receive a data set, for example from a scanner, defining the shape of an acetabulum, and to determine from the data set a specification for at least a part of the locating means.

The system may further comprise production means arranged to produce said part of the locating means according to the specification. Alternatively, or in addition, the system may further comprise a memory having a component specification for each of a plurality of components stored therein, and the processing means may be arranged, in determining the specification for at least a part of the locating means, to select at least one of the component specifications.

The system may further comprising a display screen arranged to display an image of the bone, for example the acetabulum, and user input means arranged to enable a user to identify the position of a feature on the bone. The processing means may be arranged to add data indicating that position to the data set.

The present invention further provides a method of shaping an acetabulum comprising providing locating means, providing guide means, and providing support means, locating the locating means in the acetabulum, locating the guide means against the locating means in a target position, supporting the guide means on the supporting means, removing the locating means, and shaping the acetabulum using a tool guided by the guide means.

The present invention further provides the following, all of which are provided by, and incorporated in, all of the systems described below with reference to the Figures (except where that is inconsistent), in some cases with appropriate modifications:
1. Software for planning the position of a joint prosthesis using 3D image data having the capability to calculate the ideal angle of insertion and position for the acetabular component of the joint prosthesis based on the landmarks identified from said 3D image data having the ability to automatically generate a digital file for the manufacture of a part using 3D printing, milling, rapid prototyping, or rapid manufacturing technologies, which fits into the acetabulum.
2. Software for planning the position of a joint prosthesis using 3D image data having the capability to calculate the ideal angle of insertion and position for the acetabular component of the joint prosthesis based on the landmarks identified from said 3D image data having the ability to automatically generate a digital file for the manufacture of a part using 3D printing, milling, rapid prototyping, or rapid manufacturing technologies, which fits into the acetabulum, and extending onto the ilium and or the acetabular notch.
3. Software for planning the position of a joint prosthesis using 3D image data having the capability to calculate the ideal angle of insertion and position for the acetabular component of the joint prosthesis based on the landmarks identified from said 3D image data having the ability to automatically generate a digital file for the manufacture of a part using 3D printing, milling, rapid prototyping, or rapid manufacturing technologies, which fits into the acetabulum.
4. Software for planning the position of a joint prosthesis using 3D image data having the capability to calculate the ideal angle of insertion and position for the acetabular component of the joint prosthesis based on the landmarks identified from said 3D image data having the ability to automatically generate a digital file for the manufacture of a part using 3D printing, milling, rapid prototyping, or rapid manufacturing technologies, which fits into the acetabulum, by defining the acute change in gradient that demarcates the inner and outer aspect of the acetabulum, the pinnacle of which defines the rim and also allows automated extension onto the ilium and or the acetabular notch.
5. Software for planning the position of a joint prosthesis using 3D image data having the capability to calculate the ideal angle of insertion and position for the acetabular component of a joint prosthesis based on landmarks identified from said 3D image data having the ability to automatically generate a digital file for the manufacture of a part using 3D printing, milling, rapid prototyping, or rapid manufacturing technologies, which fits into the acetabulum. The peripheral extension of said part is determined automatically by the software, which is able to determine the zenith at each point along the periphery of the acetabulum, so as to create a part, which will fit within the joint capsule without requiring extensive surgical exposure of surfaces beyond the acetabulum.
6. Software for planning the position of a joint prosthesis using 3D image data having the capability to calculate the ideal angle of insertion and position for the acetabular component of a joint prosthesis based on landmarks identified from said 3D image data having the ability to automatically generate a digital file for the manufacture of a part using 3D printing, milling, rapid prototyping, rapid manufacturing technologies which fits into the acetabulum. The periphery of said part is determined automatically by the software, which is able to determine the zenith at each point along the periphery of the acetabulum, so as to create a part, which will fit within the joint capsule without requiring extensive exposure of surfaces beyond the acetabulum, however the periphery of said part may be manually edited to extend onto or avoid regions determined by the operator.
7. Software as described above having the ability to generate a digital design (e.g. STL file) or similar digital specification for a part, as described in previous claims where said part is composed of multiple parts or components, each with different functions.
8. Three dimensional planning software as described above which permits the design and manufacture of a part having specific features and geometry oriented in relation to the axis and position of the acetabular component of a prosthetic joint as defined by the software
9. Three dimensional planning software as described above in which the designed and manufactured part is designed to fit together with or contain a fitting for either a removable prefabricated standard directing component or alternatively a removable bespoke directing component, said directing component oriented by the software in relation to the ideal axis and position of the acetabular component as defined by the software.
10. Three dimensional planning software as described above which permits the design of a part which is designed to contain a fitting for, or fit together with, either a removable prefabricated standard directing component or alternatively a removable bespoke directing component. With removable directing component removed, an opening at the base of the part allows the base of the acetabulum to be explored, and the proximity or ‘fit’ of the part with the acetabulum to be ascertained.
11. Three dimensional planning software as described above which allows the design of a part as described above, which incorporates a fitting for a directing component as described above, such that when the directing component is located in the fitting, the directing component may be used to accurately locate or position a guiding sleeve or other guiding extension, device, or assembly, for the purposes of orienting the preparation of the acetabulum by the surgeon.
12. Three dimensional planning software as described above which allows the design of a part as described above, which incorporates a fitting for directing a component as described above, such that when the directing component is located in the fitting, the directing component may be used to accurately locate or position a guiding sleeve or other guiding extension, device, or assembly, for the purposes of guiding the depth and orientation of the surgical preparation of the acetabulum, thus enabling the transfer of planning data from the planning software to the operative site.
13. Software as described above which is used to design a bespoke device that will acts as a positioning or locating guide, for a separately or remotely anchored guiding sleeve or other guiding extension, device, or assembly, for the purposes of guiding the depth and orientation of the surgical preparation of the acetabulum.
14. Software as described above which is used to design bespoke spacers, which when fitted to a standard device or range of devices, will secure said device such that it will act as a positioning or locating guide, for a separately or remotely anchored guiding sleeve or other guiding extension, device, or assembly, for the purposes of guiding the depth and orientation of the surgical preparation of the acetabulum.
15. Software as described above where the part that is designed additionally contains guiding elements or surfaces which act as a guide for the preparation of sites remote from the acetabulum, for the placement of robust screws or anchors which will anchor an assembly which supports a guiding sleeve or other guiding extension or device, such that a position defined by a directing component stabilised within the acetabulum, may be transferred to the guiding sleeve or other guiding extension, or device.
16. Software as described above capable of designing an RP (rapid prototyping) part to fit into the acetabulum and also design guiding components or surfaces within or upon said part to permit anchorage elements to be inserted into the ilium without penetrating into the reamed acetabulum
17. Software as described above in which the part additionally contains a means to guide precise placement of anchoring screws for the aforementioned assembly.
18. Software as described above in which the part additionally contains a guide or guides for precise placement of the anchoring screws of the aforementioned assembly, the anchoring screws having features that constrain lateral and or vertical positioning within said guiding or constraining features, so as to ensure precise positioning of the assembly, such that a guiding sleeve or other guiding extension or device is supported in the planned position over the acetabulum.
19. Software as described above in which some or all of the components of the assembly, or other guiding extensions, or devices, are selected from a library of said components within the software, such that a guiding sleeve or other guiding extension or device is supported in the planned position over the acetabulum.
20. Software as described above in which some or all of the components of the assembly, or other guiding extensions, or devices, are designed within the software, to be produced using similar manufacturing processes to those described in previous claims.
21. A device, which when fitted into the acetabulum projects or supports a directing component or rod, which indicates the intended axis of the acetabular component of a prosthesis.
22. A device, which when fitted into the acetabulum projects or supports a directing component or rod, which indicates the intended axis and depth of the acetabular component of a prosthesis.
23. A customised device, which when fitted into the acetabulum projects or supports a directing component or rod, which indicates the intended axis of the acetabular component of a prosthesis.
24. A customised device, which when fitted into the acetabulum projects or supports a directing component or rod, which indicates the intended axis and depth of the acetabular component of a prosthesis.
25. A device, customised or adapted to fit the acetabulum by means of customised spacers or other projecting components designed and produced using CAD CAM technology. The whole, when fitted into the acetabulum projects a directing component or rod, which indicates the intended axis of the acetabular component of a prosthesis.
26. A device, customised or adapted to fit the acetabulum by means of spacers or other projecting components, where the projection of said components is defined by planning software. The whole, when fitted into the acetabulum projects a directing component or rod, which indicates the intended axis and or depth of the acetabular component of a prosthesis.
27. A device customised or adapted to fit the acetabulum by means of customised spacers or other projecting components designed and produced using CAD CAM technology. The whole, when fitted into the acetabulum projects a directing component or rod, which indicates the intended axis and depth of the acetabular component of a prosthesis.
28. A device, which is modified or adapted by CAD CAM technology to fit into the acetabulum, such that it projects a directing component or rod, which indicates the intended axis and or depth of the acetabular component of a prosthesis.
29. A standardised drilling, preparation, or alignment guide, or range of drilling, preparation, or alignment guides, intended to fit a particular anatomical region, which is adapted to fit a specific individual by means of customised spacers or other projecting components designed and produced using CAD CAM technology.
30. A standardised drilling, preparation, or alignment guide, or range of drilling, preparation, or alignment guides, intended to fit a particular anatomical region, which is adapted to fit a specific individual by means of spacers or other projecting components, where the projection of said components is defined by planning software.
31. Software for the adaptation of a standard drilling, preparation, or alignment guide, for the acetabulum, or range of guides of different sizes and somewhat different shapes, which produces a design or specification for RP parts which when fitted to the selected standard guide, orientate the guide to a specific orientation within the acetabulum, to fit a specific individual, in a manner planned and defined in the software to allow drilling or preparation to the correct alignment and depth.
32. Software for the adaptation of a standard drilling, preparation, or alignment guide, for a particular anatomical region, or range of guides of different sizes and somewhat different shapes, which produces a design or specification for RP parts which when fitted to the selected standard guide, orientate the guide to a specific orientation, to fit a specific individual, in a manner planned and defined in the software to allow drilling or preparation to the correct alignment and depth.
33. Software for the adaptation of a standard drilling, preparation, or alignment guide, for a particular anatomical region, or range of guides of different sizes and grossly different shapes, which produces a specification for the projection of parts which when fitted or extended from the fit surface of the selected guide, orientate the guide to a specific orientation, to fit a specific individual, in a manner planned and defined in the software to allow drilling or preparation to the correct alignment and depth.
34. Software for the adaptation of a standard drilling, preparation, or alignment guide, for the head of the femur, or range of guides of different sizes and somewhat different shapes, which produces a design or specification for RP parts which when fitted within the selected standard guide, orientate the guide to a specific orientation, to fit a specific individual, in a manner planned and defined in the software to allow drilling or preparation to the correct alignment and depth.
35. Software for the adaptation of a standard drilling, preparation, or alignment guide, for the head of the femur, or range of guides of different sizes and somewhat different shapes, which produces a specification for the projection of parts which when fitted or extended from the fitting surface of the selected guide, orientate the guide to a specific orientation, to fit a specific individual, in a manner planned and defined in the software to allow drilling or preparation to the correct alignment and depth.
36. Software for planning joint replacement, which models the movement of a joint, or a joint reconstructed by means of a prosthesis, and highlights regions of impingement, between bony and or implant surfaces.
37. Software as described in the previous claim, where a specification for the production, adaptation, and or orientation of a trimming guide is produced, which makes evident the envelope of the range of movement of the joint. Said guide to be fitted using the technology described in previous claims
38. Matched drills and anchorage components designed to work together with a device designed with computer aided design or surgical planning software as described above, such that site preparation for said components may be roughly guided towards ‘safe’ areas of thick bone to secure strong anchorage away from the critical structures or surfaces.
39. Matched drills and anchorage components designed to work together with a device designed with computer aided design or surgical planning software as described above, such that site preparation for said components may be precisely constrained and guided towards ‘safe’ areas of thick bone to secure strong anchorage away from the critical structures or surfaces.
40. An assembly, or the components of such an assembly, which may be anchored to the pelvis or ilium, to support and stabilize a guiding sleeve or other guiding extension or device in place in a position determined by a part that has been fitted into the acetabulum
41. An assembly, or the components of an assembly, which may be anchored to the pelvis or ilium, to support and stabilize a guiding sleeve or other guiding extension or device in place in a position determined by a customised part that has been fitted into the acetabulum
42. An assembly, or the components of an assembly, which may be anchored to the pelvis or ilium, to support and stabilize a guiding sleeve or other guiding extension or device in place in a position determined by a part that has been fitted into the acetabulum, said part having been designed by software as described above.
43. An assembly as described above having moveable, jointed, but lockable arms
44. An assembly or the components of an assembly that is anchored into the ilium or any accessible part of the pelvis or a framework that is itself anchored to the pelvis, which by the use of interlinked components with adjustable but lockable joints may be used to position a reamer guide for preparation of the acetabulum, in a pre-planned relationship as determined by 3D planning software and transferred to the operative field by means of a customised component that fits into the acetabulum.
45. An assembly that is anchored into the ilium or any accessible part of the pelvis or the framework that is itself anchored to the pelvis which by use of the interlinked components with adjustable joints may be used to position a guide for preparation of the acetabulum, in a pre planned relationship as determined by 3D CAD CAM planning software and transferred to the operative field by means of a customised component that fits into the acetabulum. The parts of the assembly may be prefabricated, multi use parts.
46. An assembly that is anchored into the ilium or any accessible part of the pelvis or the framework that is its self anchored into the pelvis which by use of the interlinked components with adjustable joints may be used to position a guide for preparation of the acetabulum, in a pre planned relationship as determined by 3D CAD CAM planning software and transferred to the operative field by means of a customised component that fits into the acetabulum. The parts of the assembly may be either prefabricated ‘standard’ parts are rapid prototyped parts or a mixture of both.
47. A method for placing the acetabular component of a hip prosthesis in which a guiding part is fitted precisely within the acetabulum, and this guiding part is then used to position a remotely anchored guiding device or structure, which is in turn used to aid positioning of the reaming instrumentation for preparation of the acetabulum.
48. A method for placing the acetabular component of a hip prosthesis in which a defined vector for the acetabular component is transferred to a guide for axial preparation of the acetabulum.
49. A custom made device that fits within acetabulum to transfer the vector of the planned acetabular component to a remotely anchored guide which in turn permits axial preparation of the acetabulum along the intended vector, and to the intended depth.
50. A custom made validation jig that attaches to the guiding fixation rods and can provide a visual check that the implant has been placed at the correct angulation and depth within the acetabulum.
51. Software, methods, and devices as described above, but adapted for use in the preparation of alternative sites for the precise placement of bony implants.

Preferred embodiments of the invention will now be described by way of example only with reference to the accompanying drawings.

### BRIEF DESCRIPTION OF THE DRAWINGS

FIG. 1 is a diagram of a computer system arranged for use in an embodiment of the present invention;

FIG. 1 shows an image stored and displayed on the system of FIG. 1 showing the acetabulum and surrounding area of the pelvis;

FIG. 2 is a perspective view of a custom acetabular assembly according to an embodiment of the invention;

FIG. 3 is a section through the custom acetabular assembly of FIG. 2 located in the pelvis with an associated guide component;

FIG. 4 is a perspective view of a component of an acetabular assembly according to a further embodiment of the invention;

FIG. 5 is a section through the component of FIG. 4 with spacers inserted to form the whole assembly;

FIG. 6 is a partially sectioned side view of an anchorage assembly in use with the guide component of FIG. 3;

FIG. 7 is a partially sectioned side view of the anchorage assembly of FIG. 6 in use to guide shaping of the acetabulum;

FIG. 8 is a top view of the customised acetabular assembly of FIG. 2 in place in the acetabulum;

FIG. 9 is a top view similar to FIG. 8 but with the guide component of FIG. 3 in place;

FIG. 10 is a top view similar to FIG. 8 with the anchorage assembly of FIG. 6 in place;

FIG. 11 is a top view similar to FIG. 8 with the anchorage assembly still in place and the custom acetabular assembly and guide component removed;

FIG. 12 is a partially sectioned side view of an anchorage assembly according to a further embodiment of the invention;

FIG. 13 is a sectional view of part of the system of FIG. 12 during reaming of the acetabulum;

FIG. 14 is a sectional view of part of the system of FIG. 12 during checking of the reaming process; and

FIG. 15 is a sectional view of part of the system of FIG. 12 during checking of an inserted acetabular prosthetic component.

### IMAGING AND SOFTWARE PLANNING

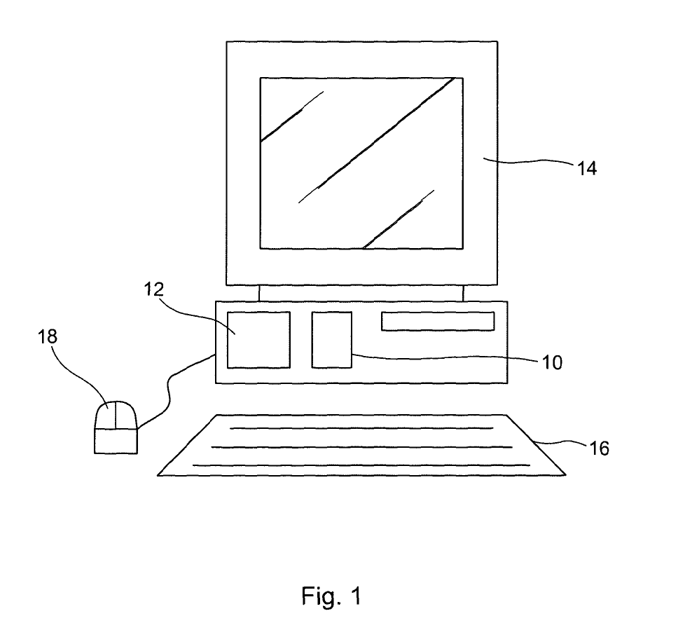

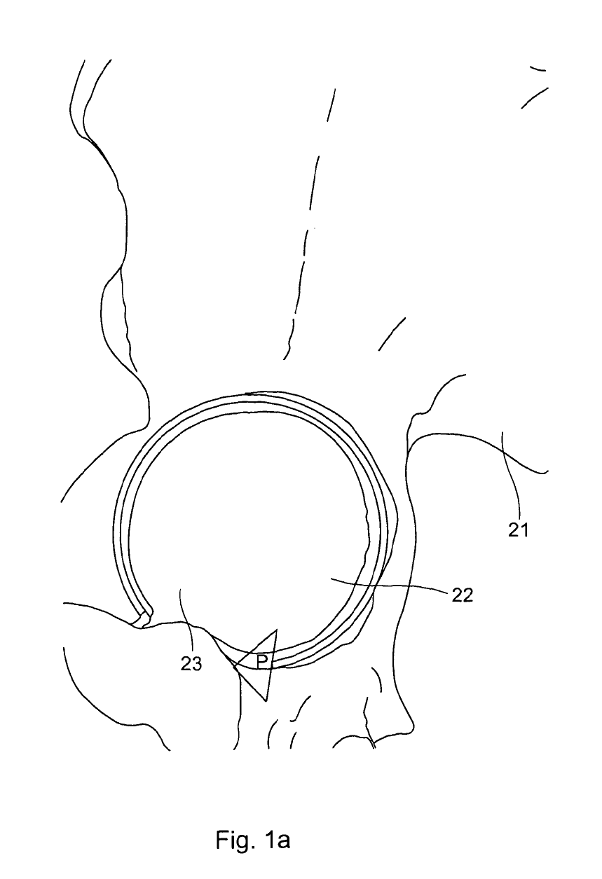

Referring to FIG. 1, a computer system comprises a memory 10, processor 12, display screen 14 and a user input in the form of a keyboard 16 and mouse 18. Using a suitable scanner system set up for 3D imaging, e.g. with Computed Tomography (CT) or Cone Beam Computed Tomography (CBCT) a 3D image dataset of the pelvis, which forms a model of the pelvis, is generated, and then stored on the memory 10 of the computer system. The system is arranged to generate an image, from the dataset, on the display screen 14 which can be viewed in an on-screen virtual environment. The memory 10 has stored on it software which is arranged to be run to enable a user to perform a number of steps to modify the dataset to add to it further data defining the position and orientation of various features of the acetabulum itself and of the prosthesis to be inserted. The software is arranged to provide access to a library of prostheses, which may be generic in form or manufacturer specific. The library includes a group of data sets defining the size and shape of various prostheses that are available for use. The library may be stored on the memory 10 of the system, or stored remotely. The library may be extended by including segmented data from CT or CBCT scans of previously treated patients, or individual prostheses, or implants. The system also has stored in memory a three dimensional model of the femur and of the femoral component of the prosthesis.

The software has visualization, modelling, measurement, and computer aided design (CAD) functionality, allowing the model of the pelvis to be viewed and analysed and a suitable prosthesis to be designed and its optimum position and orientation relative to the pelvis to be determined by a user such as a surgeon.

The software enables the segmentation of the data set, with the production of an on-screen 3D model, e.g. by surface or volume rendering, derived from the dataset by assigning a threshold value to the data.

The software enables reference planes for the pelvis to be identified, either manually or automatically, and definitions of those planes to be added to the data set. The target position of the acetabular component of a joint prosthesis in terms of the location and orientation of its central axis, its angulation about that axis, and its depth along that axis, can then be planned with respect to the reference anterior and transverse pelvic planes by the user. The definition of that target position is then added to the data set. Alternatively, if the femoral head is deemed by the surgeon or other operator to be in a suitable position, the software can also be arranged to segment or allow the segmentation of the image data of the femoral head, to identify the rotational axis of the femoral head by using e.g. a pattern recognition algorithm to approximate the rotation of the femoral head within the acetabulum.

Alternatively a healthy contralateral joint may be modelled, to allow the orientation of the joint to be mirrored to the affected side.

Having established a possible position for the acetabular component of the joint prosthesis, the software enables rotation of the femoral component along with the femur to be modelled to explore or model the range of movement provided by this orientation, enabling the detection of potential regions of impingement between bony surfaces, and, or, prosthetic components to be identified.

As shown in FIG. 1a, the surgeon (or other operator) is then able to indicate on screen the extent of the proposed surgical exposure of the acetabulum 22 and adjacent surfaces, including the ilium, of the pelvis 21, the inferior margin of the true floor of the acetabulum 22 (known radiographically as the “teardrop”); cotyloid fossa; and various parts of the acetabular rim (P) and the acetabular notch 23).

The positions of these features are added to the model data set. This positional information defines the perimeter of the surgical exposure. Within this perimeter, at operation, the surgeon will have access to the surface of the acetabulum, with potential extension onto the ilium and the acetabular notch.

All of the software described is stored in the memory 10 of the computer, but can be provided on a suitable data carrier, such as a CD, memory stick or other memory device, for backup, copying or sale.

Generating a Specification for the Acetabular Assembly

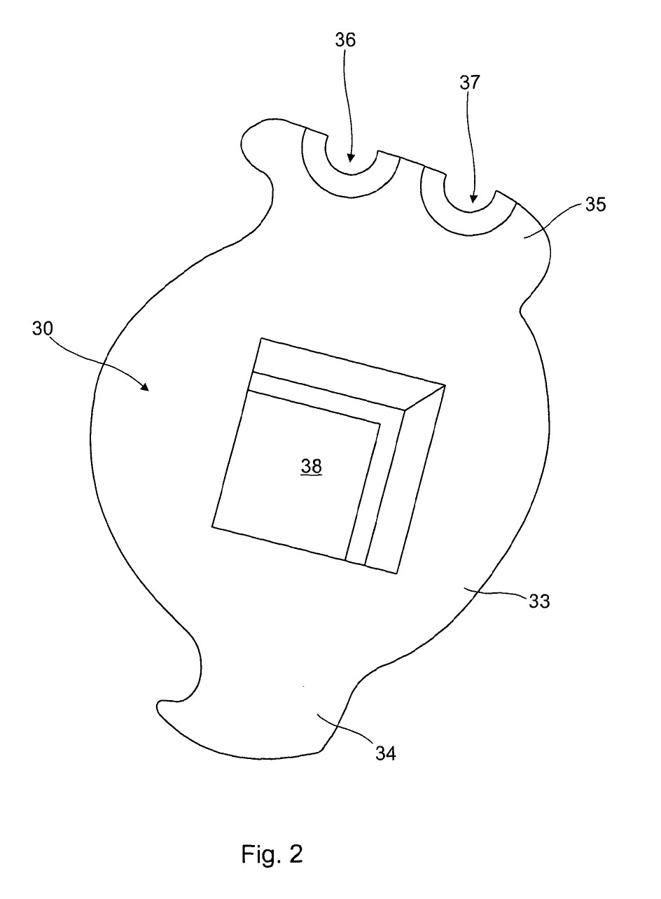

The software is arranged to use the model data, including the surface data and described perimeter to create a digital specification for a custom acetabular assembly 30, as depicted in FIG. 2, that will fit within the defined perimeter. The custom acetabular assembly 30 is arranged to be accurately located in the acetabulum and to support a guide rod 40, shown in FIG. 3. As will be described in more detail below, the guide rod 40 is used to guide the locating of an anchorage assembly which is anchored in the pelvis outside the acetabulum and, after removal of the guide rod 40 and custom acetabular assembly 30, used to control shaping of the acetabulum in preparation ready to receive the acetabular component of the prosthesis.

Referring back to FIG. 2 the bulk of the custom acetabular assembly 30 may take a number of forms, ranging from a skeletal object that fits in contact with a few carefully defined points within the acetabulum and around the perimeter, or, as in the embodiment shown in FIG. 2, a robust and bulky solid object. This object, when it is produced, will be ‘adapted’ and designed to include certain features, which will be defined in the digital specification and modelled so as to be visible on-screen, before the digital specification is used to generate a file, e.g. an ‘stl’ file, for the production of the physical custom acetabular assembly 30, using rapid prototyping or rapid manufacturing technologies such as stereolithography, CNC milling, or photo-jetting.

The software may include automated utilities that will automatically or semi-automatically describe the perimeter of the acetabulum and in turn define the shape of the custom acetabular assembly 30, perhaps with reference to the change in gradient associated with the acetabular rim, and, or a library of acetabular morphologies. The custom acetabular assembly 30 may also act as a crude or precise template or guide to adjustment or alteration of the peripheral contour of the acetabulum, to a prescription defined by the modelling of the movement of the joint, and noting the regions of impingement.

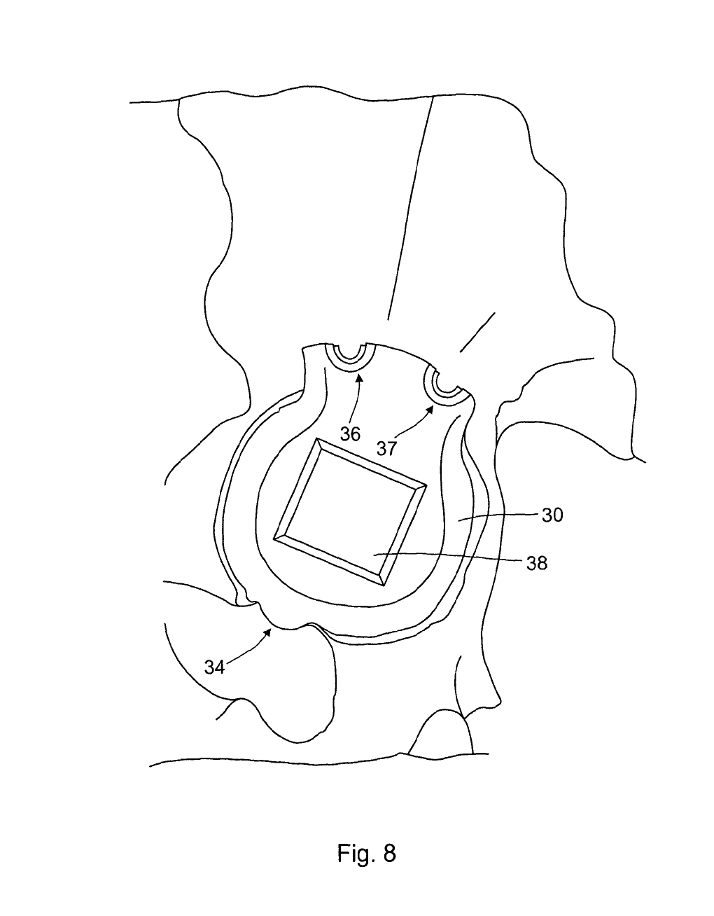

With reference to the measured and planned position of the acetabular component, the software determines the shape of the custom acetabular assembly 30. As can be seen in FIGS. 2 and 3 the custom acetabular assembly 30 has a back side 31 arranged to locate against the surface of the acetabulum 22 and a front side 32. In the embodiment of FIGS. 2 and 3 the assembly 30 has a main body 33 the back side of which has a continuous contact surface which is approximately part-spherical. On one side of the main body 33 there is a hooked locating portion 34 arranged to locate in the acetabular notch 2. This hooked locating portion 34 has back surface which is concave in the radial direction of the acetabulum and convex in the circumferential direction so that it is arranged to fit around the surface of the acetabular notch 2. On the diametrically opposite side of the main body 33 to the hooked locating portion 34 the assembly further comprises a guide portion or lip 35 arranged to extend over the rim of the acetabulum. The lip 35 extends around about a quarter of the circumference of the acetabulum and has two guide surfaces 36, 37 formed in it each defining a channel (which can be seen more clearly in FIG. 8) in which a respective threaded rod can be located as described in more detail below. The two channels are approximately parallel with each other and each extend from the front of the assembly to the back, having one end opening at the back of the assembly where it locates against the surface of the pelvis. In the main body 33 of the assembly a recess 38 is formed. This recess 38 has a standardised configuration, in this case being square in cross section and stepped, such that a standard guiding component 40 can be located in it.

The guiding component 40 comprises a male part 42 that fits within the recess 38, and a guiding rod 43 which is rigidly connected to the male part 42. The guiding component may be precisely seated within the recess 38 so that correct alignment of the custom acetabular assembly 30 ensures correct alignment of the guiding component 40 with respect to the acetabulum. The position of the guiding rod 43 reflects the optimum vector for the mechanical preparation of the acetabulum. Specifically the longitudinal axis of the guiding rod 43 is parallel to the axis of rotation of the reaming tool, which is also the axis along which the reaming tool is moved during the reaming process to set the depth of the shaped acetabulum. Furthermore, suitable markings along the length of the guiding rod 43, which may correspond to markings on the instrumentation used to prepare the acetabulum, may indicate the ideal depth for the preparation.

The recess 38 extends right through the assembly 30, thereby forming an aperture through the assembly 30, and is designed such that the bony base of the acetabulum may be visualised and explored with an instrument by removing the guiding component 40, so as to be able to verify the fit of the assembly 30.

Thus at the time of surgery, the custom acetabular assembly 30 may be positioned in the acetabulum with no more than normal exposure of the site, save perhaps for slight extension on to the ilium and beneath the transverse acetabular ligament. With the custom acetabular assembly 30 in place the recess 38 allows the seating of the custom acetabular assembly 30 to be verified before the guide component 40 is inserted. With the guide component 40 inserted, the guide rod 43 is in line with the correct vector (which is commonly referred to in terms of inclination and version angles) for movement of the reaming tool during the reaming process.

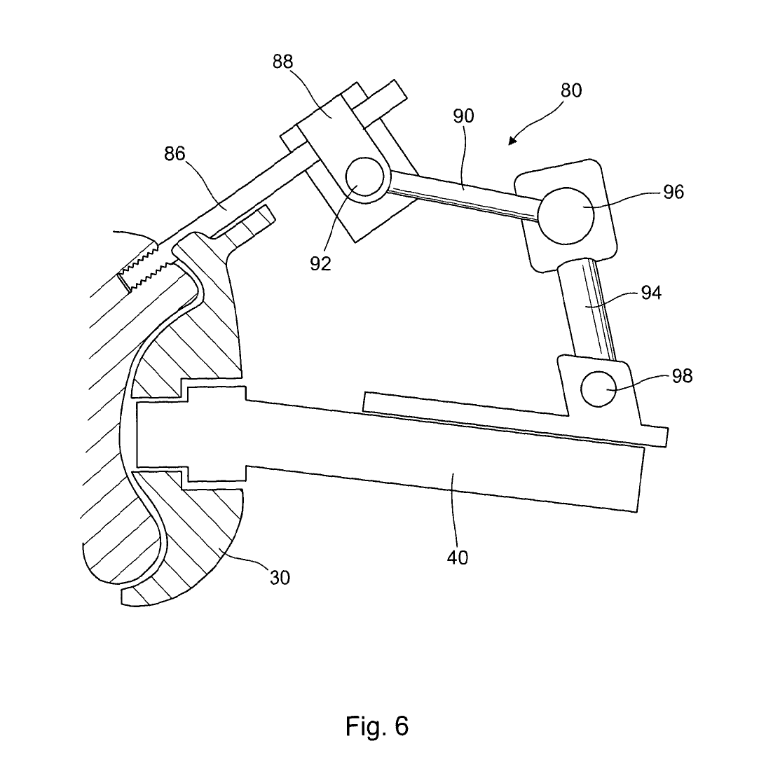

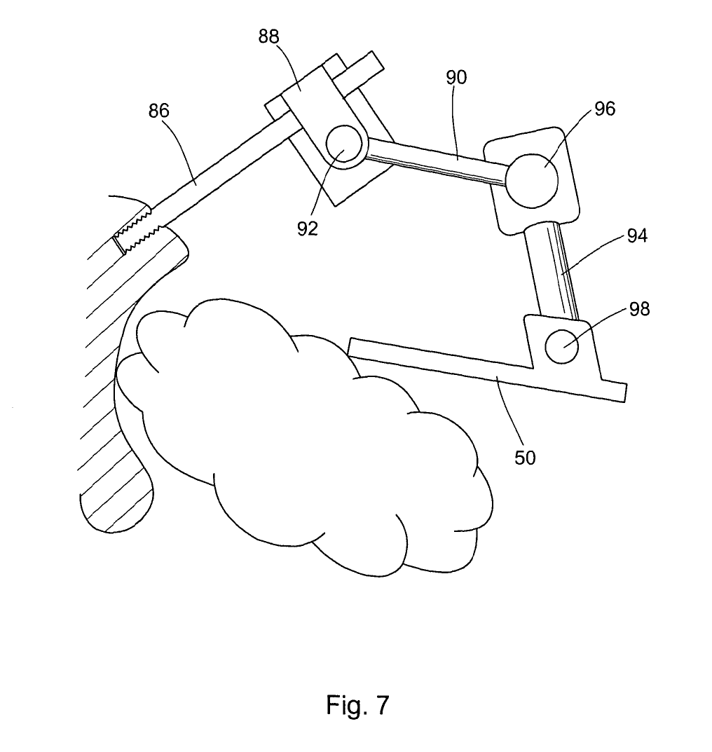

With the intended vector for the reaming process so determined, the next step is to provide a reaming guide 50 as shown in FIG. 6. However it will be appreciated that any guidance provided must allow for reaming of the acetabulum with the custom acetabular assembly 30 removed.

Custom Acetabular Assembly and Customised Standard Acetabular Assembly

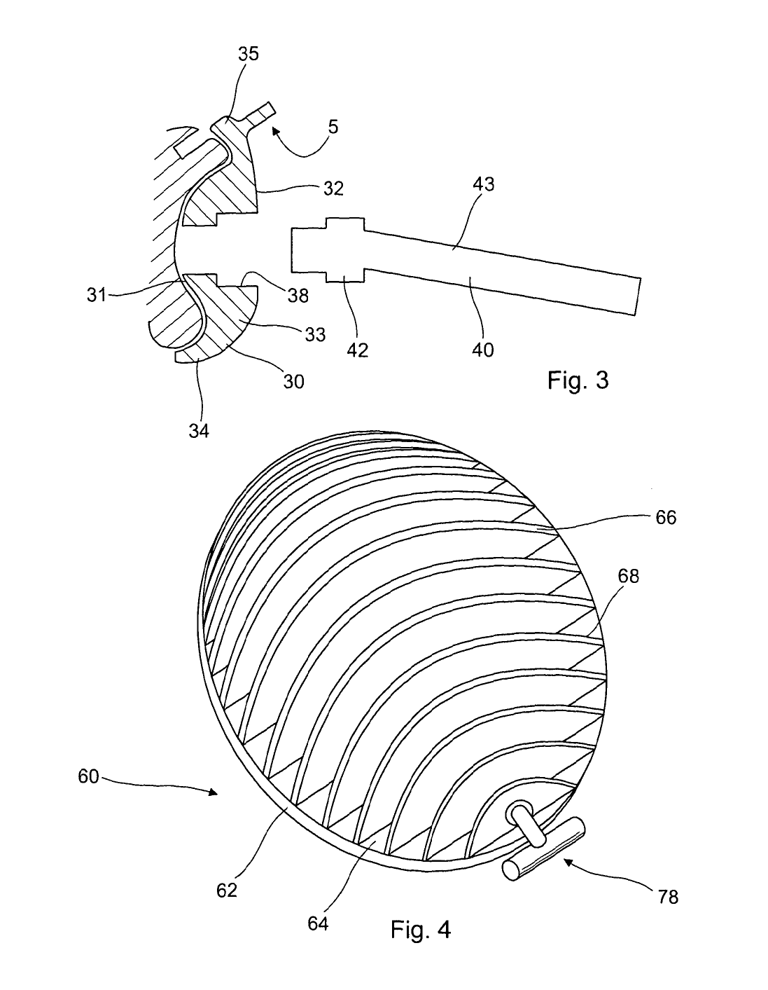

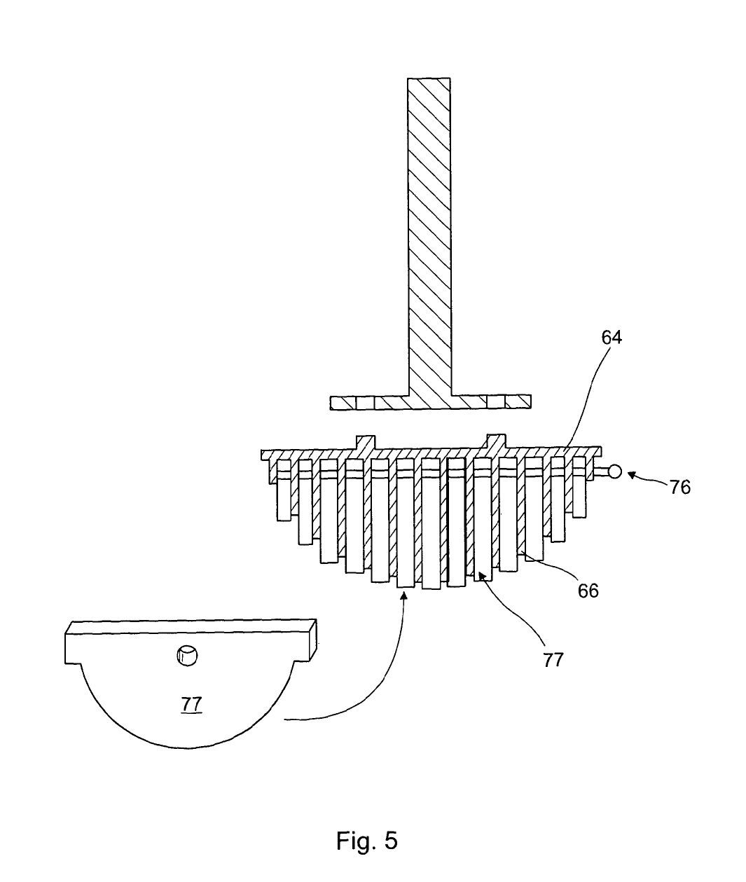

The particular topography of the acetabulum is such that it is grossly similar from patient to patient; which is why standard prostheses have evolved in different sizes, but similar shapes. Another embodiment of the invention provides a customised standard acetabular assembly which comprises a template 60 as shown in FIGS. 4 and 5. In this embodiment, the main body 62 of the template 60 comprises a base in the form of a plate 64, which is approximately circular, forming the front surface of the main body, with a series of parallel plates or fins 66 projecting from the back surface of the plate 64 approximately perpendicular to the plate 64. Each fin 66 is approximately part circular in shape having a curved rear edge 68, with the fins varying in size so that their rear edges 68 are arranged to locate against the inner surface of the acetabulum. This template component therefore forms a standard acetabular assembly, having properties similar to those of the custom acetabular assembly 30 described above. It may include a hooked locating portion to locate in the acetabular notch and a guide portion having guide surfaces formed on it similar to those of the custom acetabular assembly 30. However referring to FIG. 5 in this embodiment, the template acetabular assembly 60 can be customised to fit a particular acetabulum by the insertion of a series of spacers 77 into the gaps between the fins 66. The spacers are each as thick as the gaps between the fins 66 and are of the same general shape as the fins 66, but each one is arranged to project slightly above the curved edges of the fins between which it fits so as to modify the shape of the curved surface against which the assembly will fit, thereby to customize the shape of the assembly to match a particular acetabulum.

The software is used to generate, from the model data set for the particular acetabulum, a spacer set design specification of a set of customised spacers 77, that, when they have been produced, are fitted to the standard acetabular assembly to allow it to be positioned precisely into a planned position. The manufacturing of the spacers 77 from the design specification may also take place using rapid prototyping or rapid manufacturing technologies. However, as the size of the spacers 77 is much reduced, they may be produced using simpler technologies, e.g. CNC milling or as in the depicted embodiment, laser cutting of sheet materials.

In the embodiment of FIGS. 4 and 5 the standard acetabular assembly has the general shape of a ‘finned’ object that may be fitted within the acetabulum. The fins 66 project from the base 64 to form spaces between them into which the laser cut spacer forms 77 may be positioned. The laser cut forms are retained in place by a locking pin 78 which passes through aligned holes in each of the fins 66 and each of the spacers 77. The position of the holes in the spacers 77 is defined as part of the design specification of the spacers as it determines their final position in the standard acetabular assembly. The upper surface of the object has a recess formed in it, not shown in the drawings, designed to retain a guiding component as described above in the embodiment of FIGS. 2 and 3. The base 64 has locating lugs 79a on its rear face which are arranged to engage with apertures 79b in a mounting plate on the end of the locating rod. This enables the locating rod to be removably connected to the acetabular assembly in a fixed position and orientation relative to it.

In this embodiment, the planning software incorporates a library of design specifications for a set of standard acetabular assemblies, and is arranged to select one of them on the basis of the data set which is the closest fit to the imaged acetabulum. The software is then arranged to generate the digital specification for the spacers 77 to be inserted into it on the basis of the image data set for the acetabulum. The system further comprises a laser cutter or other production system that is arranged to produce the spacers 77 to the digital specification.

While flat planar spacers are used in the embodiment described above, other shaped spacers can also be used. For example the spacers could comprise a set of rods of different lengths arranged to locate in respective holes in the back of the acetabular template. The rods can form an array providing an array of contact points for contacting the acetabulum. Alternatively a smaller number of rods, for example six or eight could be used. Similarly in a system with flat spacers like those of FIG. 5 the number of spacers could be significantly less, provided there are at least two, though obviously the more there are the better the device can be fitted to the acetabulum.

Anchorage Assembly

Referring to FIG. 6, as described above, the system further comprises an anchorage assembly 80 comprising of a system of rods 82 and lockable ‘universal’ joints 84 that is used to support and stabilise the reaming guide 50. The anchorage assembly comprises a pair of threaded rods 86 arranged to be screwed into the ilium superior to the acetabulum where the ilium is a thick dense bony structure, which may be conveniently used to anchor the anchorage assembly 80. A sliding support 88 is slidably mounted on the threaded rods 86, and has one end of a first connecting rod 90 connected to it by a universal joint 92. A second connecting rod 94 is connected to the first by a further universal joint 96 and the reaming guide 50 is connected to the second connecting rod 94 by means of a further universal joint 98. All of the universal joints 92, 96, 98 are releasably lockable so that the position of the reaming guide 50 can be adjusted by locating it against the guide rod 43 to locate it in the desired position relative to the acetabulum.

The software allows the best position for the screw-retained anchorage assembly 80 to be determined. The acetabular assembly is provided with guiding surfaces 76, 77 as described above which are arranged, i.e. positioned relative to the recess 38 such that the site preparations for the robust threaded rods 86 that anchor the anchorage assembly to the ilium may be sited in dense bone, and at an angle that will avoid impingement of the threaded rods 86 into the reamed acetabulum.

The software may contain a library of the components from which the anchorage assembly 80 is made up, the library containing a set of data for each component defining its dimensions, such that by planning the approximate position of the threaded rods, the reaming guide may be correctly aligned and positioned by the guide rod, and then locked into position by the anchorage assembly 80, such that when the custom acetabular assembly is removed, the reaming guide remains correctly aligned, fixed in space.

In an alternative embodiment, the software may determine a precise position for the anchorage assembly, such that the custom acetabular assembly provides more rigidly constraining guidance for the threaded rods, and a fixed or somewhat adjustable anchorage assembly, may be provided. In this case, the custom acetabular assembly may be designed such that it incorporates a reaming guide which can be exactly located by the location of the custom acetabular assembly. The anchorage assembly can then be anchored in the bone and then connected to the reaming guide, and then locked (if it is adjustable) so as to maintain the reaming guide in position. The reaming guide may then be detached from the main body of the custom acetabular assembly, which can then be removed.

With the reaming guide suspended in place, site preparation can proceed for the acetabular component, along the planned axis/vector, and to a depth defined in the software in relation to a marking on the RG.

The acetabular component is then impacted into the prepared site, (or if necessary screwed into position), aligning the impactor with the reaming guide, and checking the relationship of the acetabular component with the reaming guide before finally detaching it from the acetabular component The correct depth of seating may then be established by fitting a ‘verification jig’ into the impacted acetabular component, and checking the position according to the position and markings on the reaming guide.

Thus in the embodiments described the principal purpose of the CAD process and the production of the custom acetabular component is the positioning of the guide rod.

Referring to FIGS. 8 to 11 the process of shaping the acetabulum and inserting an acetabular prosthetic component will now be described. Firstly, referring to FIG. 8, once the custom acetabular component 30 has been designed from the image data and produced, it is then located in the acetabulum with the hooked locating portion 34 located in the acetabular notch and the guide surfaces 36, 37 aligned with points on the pelvis offset from the acetabulum. Because it has been designed to fit the acetabulum, the custom acetabular component 30 should be a good fit, and this can be checked by looking through the recess 38.

Referring to FIG. 9, the guide component 40 is then located relative to the custom acetabular component 30 by inserting its male part 42 into the recess. This results in the guide rod 43 being aligned with (i.e. parallel to) the central axis of the acetabulum which will be used as the axis for the reaming tool (or other shaping tool) along which it is moved to shape the acetabulum and also the axis with which the acetabular prosthetic component will be aligned. The threaded rods 86 are also attached to the bone, in respective positions defined by the guide surfaces 36, 37.

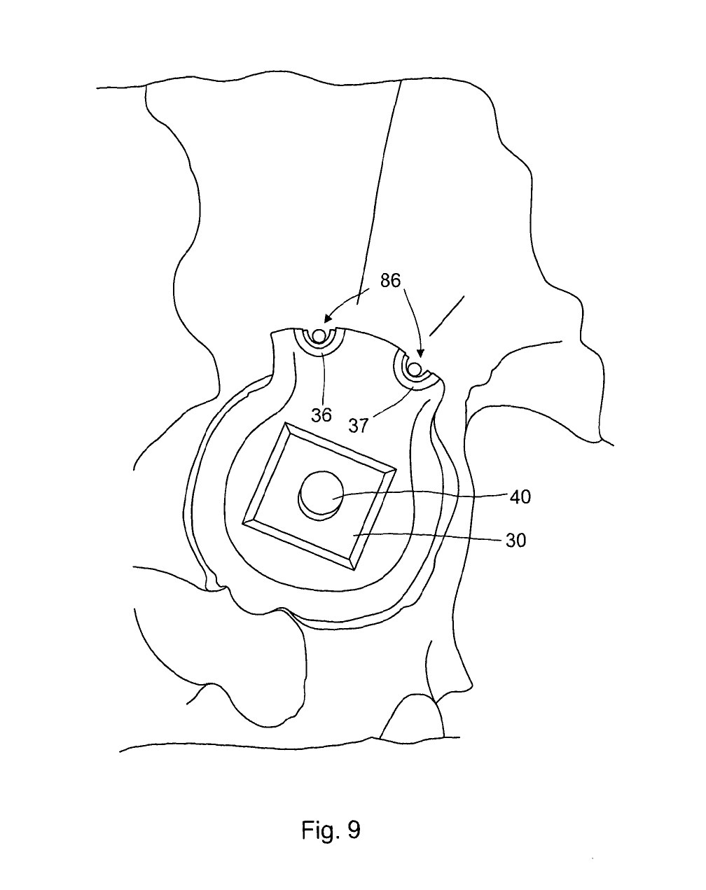

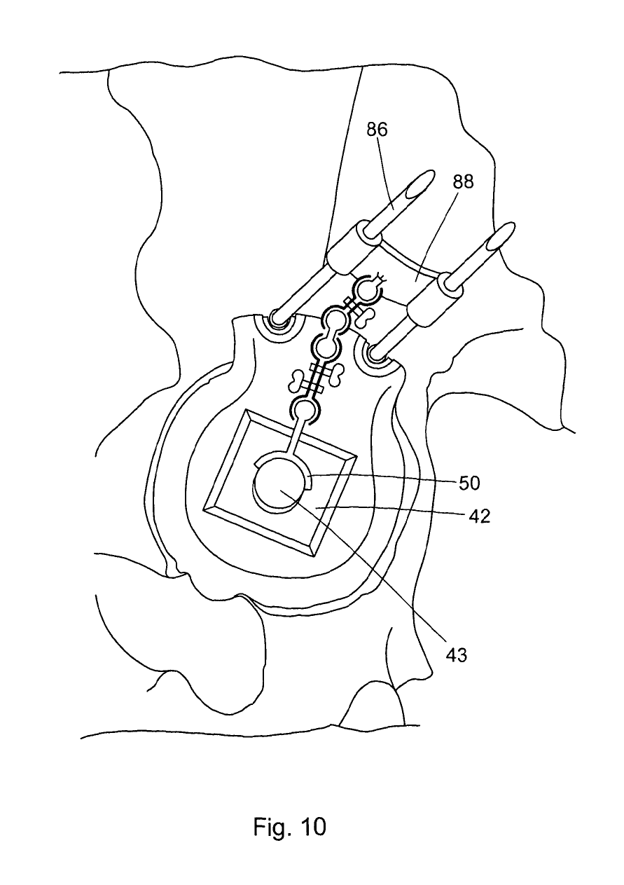

Referring to FIG. 10, the rest of the anchorage assembly is connected to the threaded rods by sliding the sliding support 88 onto the threaded rods with the connecting rods 90, 94 and reaming guide 50 connected to it. The reaming guide 50 is then moved into contact with the guide rod 43 and then the reaming guide is locked in position by locking the universal joints 92, 96, 98. Cooperating stop surfaces on the guide member 40 and the reaming guide 50 can be arranged to locate the reaming guide in the axial direction relative to the guide member 40, so that the position of the reaming guide 50 is fixed in three dimensions relative to the acetabulum.

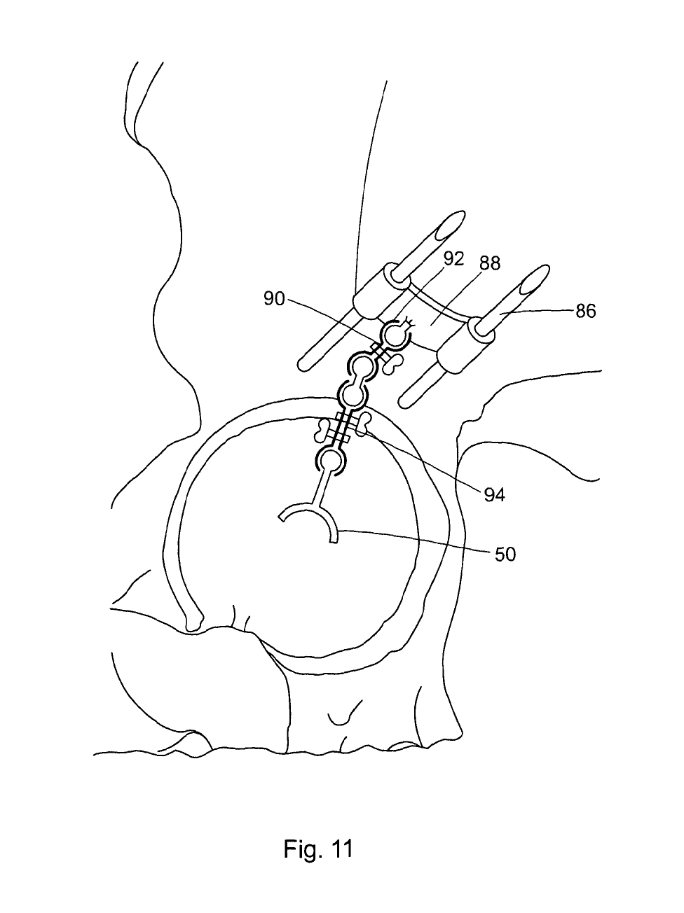

Referring to FIG. 11, the guide member 40 and custom acetabular component 30 are then removed from the acetabulum leaving the anchorage assembly and reaming guide in place to guide a reaming tool during reaming of the acetabulum. In a modification to the embodiment shown, the threaded rods 86 may have a locating device, such as a stepped diameter or other projection, that locates the sliding support 88 in a fixed position on the threaded rods by limiting its downward movement along them. This allows the sliding support 88, when the universals joints have been locked to lock the shape of the anchorage assembly, to be lifted off the threaded rods to allow removal of the acetabular component 30 and guide member 40, before being replaced in exactly the same position to guide the reaming process.

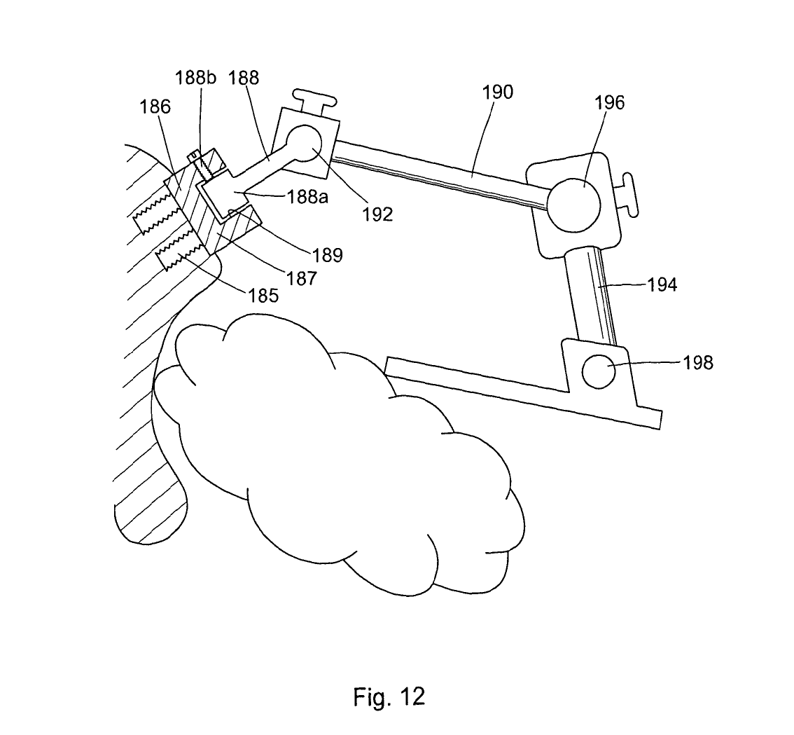

Referring to FIG. 12, in a further embodiment the anchorage assembly comprises the same basic components as that of FIG. 6, with corresponding components indicated by the same reference numerals increased by 100. However the threaded rods and sliding support are replaced by a mounting socket 186 and mounting rod 188 respectively. The mounting socket is arranged to be secured to the bone, for example by threaded fastenings 185 and includes a body 187 which has a recess 189 formed in it which forms the socket. The mounting rod 188 has a boss 188a on one end which is arranged to be a snug fit within the socket. A locking device, such as a screw 188b, is arranged to lock the boss 188a on the socket to lock the mounting rod in position relative to the socket, or to release it to allow the mounting rod 188 and the rest of the anchorage assembly to be removed and then replaced.

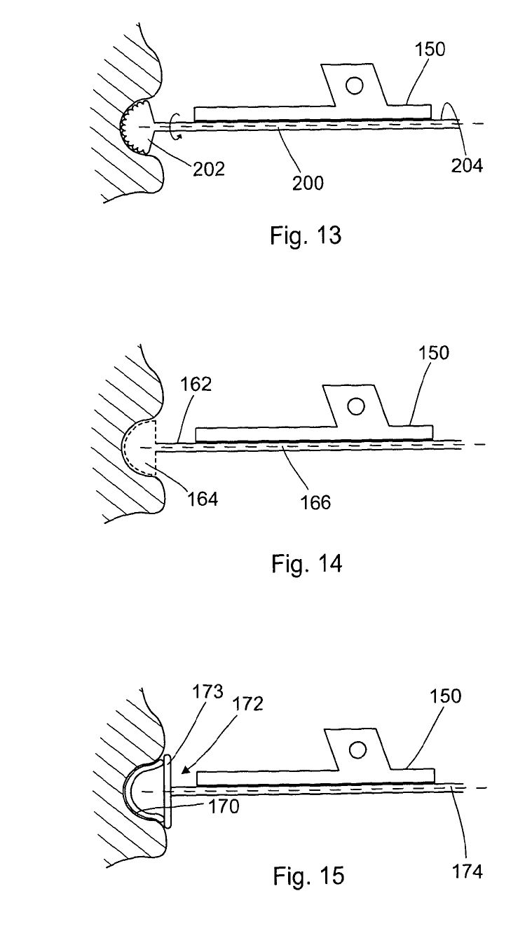

Referring to FIG. 13, once the custom acetabular assembly and the guide element have been removed from the acetabulum, and the anchorage assembly and reaming guide 150 replaced, the reaming guide 150 is used to guide a reaming tool 200. The reaming tool comprises a cutting head 202 which is mounted on a shaft and arranged to rotate about a reaming axis as the tool is moved forwards to shape the acetabulum. The reaming tool includes a guide surface 204 which is parallel to the reaming axis and is arranged to engage with the reaming guide 150 during reaming to guide the reaming tool. The reaming tool may further comprise a stop arranged to limit its axial movement along the reaming axis relative to the guide 150, so that the final position of the reaming tool and hence also of the surface of the shaped acetabulum, is fixed in three dimensions by the reaming guide 150.

Referring to FIG. 14, when the reaming is completed, a checking tool 160 is aligned with the reaming guide 150. The checking tool also includes a guide surface 162 which is arranged to engage with the reaming guide 150, and a shaped checking head 164 which is shaped to fit within the shaped acetabulum and hollow with a set of apertures through it, so that the surface of the acetabulum can be inspected through the apertures when the checking head is located in the acetabulum. Again the checking tool 160 may have a stop surface arranged to cooperate with a stop surface on the reaming guide 150 so as to locate the checking tool in three dimensions relative to the target position of the prosthetic acetabular component.

If the checking confirms that the shaping of the acetabulum has been performed correctly the checking tool is removed and the prosthetic acetabular component 170 can be put in place as shown in FIG. 15, together with a further implant checking device 172, which comprises a plate 173 arranged to be located on the prosthesis 170 and to lie in a plane perpendicular to the central axis X of the prosthesis, which in this case is aligned with the reaming axis. The checking device further comprises a position checker 174 which includes a guide surface 176 arranged to cooperate with the reaming guide 150 to locate the position checker in two dimensions so as to align it with reaming axis, and may also comprise a stop surface arranged to locate it in the axial direction relative to the reaming guide during checking. The position checker 174 is then used to check the position of the plate 173 and hence of the prosthesis 170.

It will be appreciated that the reaming guide 150, the reaming tool 200, the checking tool 160 and the implant checking device 172 can be provided as a set which are all arranged to be used together in a single operation.

Although in the embodiments described the reaming guide is stabilised by a system of rods affixed to the ilium, any suitably stable fixation, may be used for this purpose.

Accuracy may be improved, and the process rendered more predictable by the use of instrumentation which is specifically designed for the guided procedure, and which may be represented in the CAD planning software such that e.g. the dimensions and depth limits of drills and reamers may be visualised in the computer planning stages.

Revision Hip Replacement/Resurfacing Surgery

This invention is also applicable to patients that are considered for revision hip replacement/resurfacing surgery. For this purpose, in a further embodiment similar to that described above, a custom acetabular assembly, (or customised standard acetabular assembly) may be fabricated to fit within the existing acetabular component. As prostheses can give rise to considerable artefact when imaged with CT or CBCT apparatus, a virtual replica of the fitted acetabular component, from a virtual library of prostheses within the software may be registered, using e.g. a voxel based registration technique, to allow production of the custom acetabular assembly or of the spacers for a customised standard acetabular assembly, despite the presence of artefact in the original data set.

1. Apparatus for guiding a shaping tool for shaping an acetabulum, the apparatus comprising locating means arranged to be located in the acetabulum, guide means for guiding the shaping tool, and support means for supporting the guide means, wherein the guide means is arranged to locate against the locating means in a target position, and the support means is arranged to support the guide means in the target position after removal of the locating means.

2. Apparatus according to claim 1 wherein the locating means comprises a body portion arranged to fit within the acetabulum.

3. Apparatus according to claim 1 or claim 2 wherein the locating means comprises a locating portion arranged to locate in the acetabular notch.

4. Apparatus according to any foregoing claim wherein the locating means includes a support guide arranged to guide location of the support means.

5. Apparatus according to any foregoing claim wherein the locating means comprises a removable locating member against which the guide means is arranged to be located.

6. Apparatus according to claim 5 when dependent on claim 2 wherein the body portion and the locating member comprise respective mounting means arranged to removably mount the locating member on the body portion.

7. Apparatus according to any foregoing claim wherein the locating means comprises a body and a plurality of spacer elements arranged to be supported on the body and to locate in the acetabulum.

8. Apparatus according to claim 7 wherein the spacer elements are each formed from a flat sheet of material.

9. Apparatus according to claim 7 or 8 further comprising locking means arranged to lock the spacer elements in fixed positions relative to the body.

10. Apparatus according to any foregoing claim wherein the support means includes an anchor arranged to be anchored in the pelvis.

11. Apparatus according to any foregoing claim wherein the support means comprises a plurality of components which are movable relative to each other and locking means arranged to lock the components in a fixed position relative to each other.

12. Apparatus according to claim 11 when dependent on claim 10 wherein the plurality of components are separable from the anchor.

13. Apparatus according to claim 12 wherein the plurality of components are separably connected to the anchor by means of a connection mechanism which is arranged to locate the plurality of components relative to the anchor in three dimensions.

14. A system for producing an apparatus according to any foregoing claim, the system comprising processing means arranged to receive a data set defining the shape of an acetabulum, and to determine from the data set a specification for at least a part of the locating means.

15. A system according to claim 14 further comprising production means arranged to produce said part of the locating means according to the specification.

16. A system according to claim 14 or claim 15 further comprising a memory having a component specification for each of a plurality of components stored therein, wherein the processing means is arranged, in determining the specification for at least a part of the locating means, to select at least one of the component specifications.

17. A system according to any of claims 14 to 16 further comprising a display screen arranged to display an image of the acetabulum and user input means arranged to enable a user to identify the position of a feature on the acetabulum, wherein the processing means is arranged to add data indicating that position to the data set.

18. Apparatus for locating an object in a desired position relative to a bone, the apparatus comprising a support body, locating means arranged to locate the object relative to the support body, and a plurality of spacer members arranged to be attached to the support body and each having a contact surface thereon arranged to contact the bone thereby to locate the support body relative to the bone.

19. Apparatus according to claim 18 wherein the spacer elements are each formed from a flat sheet of material.

20. Apparatus according to claim 18 or 19 further comprising locking means arranged to lock the spacer elements in fixed positions relative to the body.

21. A method of producing a locating means arranged to be located in the acetabulum, the method comprising scanning the acetabulum to generate a data set defining the shape of the acetabulum, determining from the data set a specification of the locating means, and producing the locating means to the specification.

22. A method according to claim 21 wherein the specification defines the shape of any one or more components of the apparatus of any of claims 1 to 13 or 18 to 20.

23. A method of shaping an acetabulum comprising providing locating means, providing guide means, and providing support means, locating the locating means in the acetabulum, locating the guide means against the locating means in a target position, supporting the guide means on the supporting means, removing the locating means, and shaping the acetabulum using a tool guided by the guide means.

24. A data carrier carrying data arranged, when run on a computer, to cause the computer to operate as a system according to any of claims 14 to 17.

25. Apparatus for guiding a shaping tool for shaping an acetabulum substantially as described herein with reference to any one or more of the accompanying drawings.

26. A system for producing an apparatus for guiding a shaping tool substantially as described herein with reference to any one or more of the accompanying drawings.

27. Apparatus for locating an object in a desired position relative to a bone substantially as described herein with reference to any one or more of the accompanying drawings.
